(19) United States
(12) Patent Application Publication
(10) Pub. No.: US 2015/0182425 A1
(43) Pub. Date: Jul. 2, 2015

(54) ANTIPERSPIRANT COMPOSITION WITH A MINERAL CLAY EMULSIFIER

(71) Applicant: The Dial Corporation, Scottsdale, AZ (US)

(72) Inventors: Haiyan Ge, Valencia, CA (US); Amy Tsotsos, Scottsdale, AZ (US)

(21) Appl. No.: 14/141,633

(22) Filed: Dec. 27, 2013

(51) Int. Cl. A61K 8/11; A61K 8/25; A61Q 15/00; A61K 8/26

(52) U.S. Cl. CPC A61K 8/11; A61K 8/26; A61K 8/25; A61Q 15/00

(57) ABSTRACT

Materials and apparatus are provided that exhibit increased efficacy. The apparatus includes an antiperspirant composition with a mineral clay emulsifier. The antiperspirant composition includes a water-in-oil emulsion. The water-in-oil emulsion includes a water-phase component that includes an active antiperspirant ingredient and a carrier ingredient. The water-in-oil emulsion also includes an oil-phase component that includes a mineral clay emulsifier to stabilize the water-phase component and the oil-phase component.

### FIELD OF THE INVENTION

The present invention generally relates to a composition to reduce precipitate formation on fabric, and more particularly relates to an antiperspirant composition with a mineral clay emulsifier.

### BACKGROUND OF THE INVENTION

Antiperspirants are personal care products that are used to prevent or reduce perspiration and odors resulting from perspiration, particularly perspiration from a person's underarm. Such antiperspirants may interact with sweat, sebum, and detergent ingredients to form precipitates on fabric. The precipitates may turn yellow with time leaving stains on fabric such as clothing. Such staining may be unsightly and embarrassing.

Accordingly, it is desirable to have an antiperspirant composition that results in less staining of a fabric surface. Additionally, it is desirable to have an antiperspirant composition that has improved application aesthetics such as a reduced sensation of stickiness, easy gliding during application and an improved sensation of lubrication. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

An antiperspirant composition with a mineral clay emulsifier includes a water-in-oil emulsion. The water-in-oil emulsion includes a water-phase component that includes an active antiperspirant ingredient and a carrier ingredient. The water-in-oil emulsion also includes an oil-phase component comprising a mineral clay emulsifier to stabilize the water-phase component and the oil-phase component.

An antiperspirant product includes a container and an antiperspirant composition housed within the container. The antiperspirant emulsion comprises an active antiperspirant ingredient disposed within a water-phase component of a water-in-oil emulsion. The antiperspirant composition also includes a carrier ingredient disposed within the water-phase component. The antiperspirant composition also includes a mineral clay emulsifier disposed within an oil-phase component of the water-in-oil emulsion. The antiperspirant composition also includes a wetting agent disposed within the oil-phase component, the wetting agent dispersing the mineral clay emulsifier within the oil-phase component.

A method for making a water-in-oil emulsion antiperspirant composition with a mineral clay emulsifier includes mixing an active antiperspirant ingredient with a carrier ingredient. The method also includes mixing a number of oil-phase ingredients, the oil-phase ingredients including a mineral clay emulsifier, a wetting agent, an emollient, a structurant, or combinations thereof.

### BRIEF DESCRIPTION OF THE DRAWINGS

The present invention will hereinafter be described in conjunction with the following drawing figures, wherein like numerals denote like elements, and

FIG. 1 is a diagram of an example of applying an antiperspirant composition with a mineral clay emulsifier according to the principles described herein.

FIG. 2 is a flowchart of a method for making a water-in-oil emulsion antiperspirant composition according to the principles described herein.

FIG. 3 is a diagram illustrating stain formation using the antiperspirant composition with a mineral clay emulsifier according to the principles described herein

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

As described above, perspiration is a common occurrence in most individual's lives. Perspiration itself and odors emanating from the perspiration can be uncomfortable, unsightly, and embarrassing. To combat perspiration, an individual may use an antiperspirant substance to reduce the perspiration itself and odors emanating from the perspiration. Such antiperspirant substances include an active ingredient. The active ingredient may reduce perspiration, perspiration odor, perspiration wetness, or combinations thereof. However, in some examples, the active ingredient may interact with detergent used to launder clothing. The interaction may result in precipitates that form on the clothing fabric. Such precipitates may also form due to an interaction between the active ingredient and other ingredients in the antiperspirant substance, sweat and sebum. Such precipitates may turn yellow in time, leaving a stain on the fabric. For example, antiperspirant compositions applied to an underarm may interact with laundry detergent to form yellow stains on the armpit region of a shirt. These yellow stains may be both unsightly and embarrassing for an individual. The stains may also reduce the lifecycle of an article of clothing.

Antiperspirants may come in various forms. For example, some antiperspirants are invisible solid stick antiperspirants, clear gel antiperspirants, and roll-on antiperspirants. Some of these antiperspirant types may exhibit application aesthetics that lead to a less than satisfactory user experience. For example, some antiperspirant substances may feel “sticky” and may be difficult to apply. Moreover, various types of antiperspirant may leave residues on the skin surface during use.

Accordingly, the principles described herein provide a composition for reducing precipitate formation on fabric, which may result in reduced yellow staining of the fabric. For example, an antiperspirant composition as will be described below may include a mineral clay emulsifier that may encapsulate the active antiperspirant ingredient. Such encapsulation may reduce the ability of the active antiperspirant ingredient to polymerize and may reduce the ability of the active antiperspirant ingredient to interact with laundry detergent, sweat, sebum soils, or other compounds. The reduced interaction may reduce the propensity of particulate formation, thus reducing yellow staining. Moreover, the clay particles of the mineral clay emulsifier may be easily washed off again reducing the probability of an interaction between the active antiperspirant ingredient and other compounds. The use of a mineral clay emulsifier may be a unique aspect of the antiperspirant composition described herein.

Additionally, the principles described herein provide an antiperspirant composition that has improved aesthetics such as a less sticky feel and that has an improved gliding motion during application. For example, the antiperspirant composition as described below may be a pickering water-in-oil emulsion that may leave less white residue and may provide pleasing application aesthetics such as a “gliding” motion.

Accordingly, the antiperspirant composition disclosed herein may prevent precipitate formation on fabric, provide improved aesthetics, all while maintaining an ability to reduce perspiration wetness and odors to leave the consumer with an overall satisfactory experience.

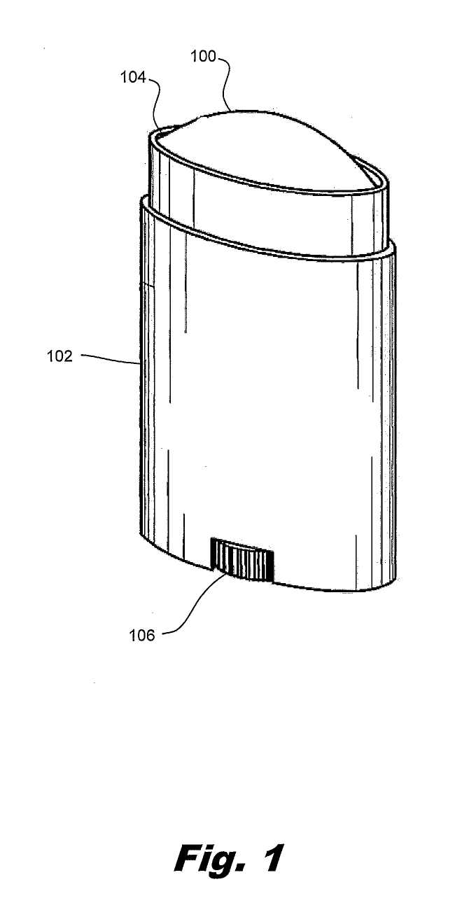

Turning now to the figures, FIG. 1 is a diagram of an example of applying an antiperspirant composition (100) with a mineral clay emulsifier according to the principles described herein. In this example, the antiperspirant composition (100) may be held within a container (102) that has an opening (104) that allows the antiperspirant composition (100) to be pushed out of the container (102) and onto a surface. For example, the solid antiperspirant composition (100) may extend beyond the opening (104) of the container (102) and be applied to skin. As indicated in FIG. 1, the antiperspirant composition (100) may be approximately dome-shaped and may be applied to skin. For example, the antiperspirant composition (100) may be applied to an underarm. The container (102) may also include a dispenser to dispense the antiperspirant composition (100). For example, the antiperspirant composition (100) may be pushed out of the opening (104) by rotating a dial (106), or a knob, positioned at the bottom of the container (102). As the dial (106) is rotated, a platform internal to the container (102) moves up and pushes the solid antiperspirant composition (100) with it.

Any appropriate type of container (102) may be used to hold and apply the antiperspirant composition (100) to skin. For example, a container (102) with an oval cross section may be used to hold the antiperspirant composition (100). Also, a container (102) with a more cylindrical cross section may be used in some examples. The container (102) may have any appropriate shape in accordance with the principles described herein. In some examples, the container (102) may include a protective cover.

During use, a top of the container (102) may be removed and the antiperspirant composition (100) exposed. Via the container (102), a user may apply the antiperspirant composition (100) to skin by rubbing the antiperspirant composition (100) across the skin.

The antiperspirant composition (100) may be a water-in-oil emulsion. In other words, the antiperspirant composition (100) may include a number of phase components that are otherwise immiscible. More specifically, the antiperspirant composition (100) may include a water-phase component and an oil-phase component. The water-phase component may include an active antiperspirant ingredient and a carrier ingredient. In some examples, the antiperspirant composition (100) may be a pickering emulsion. That is, the antiperspirant composition (100) may use solid particles to stabilize the water-phase component and the oil-phase component. As will be described below, the solid particle may be a mineral clay emulsifier. A water-in-oil emulsion antiperspirant composition (100) may be beneficial in that it may leave less white residue on a skin surface and may improve application aesthetics. In other words, the water-in-oil antiperspirant composition (100) may feel smoother during application.

The active antiperspirant ingredient may be any active ingredient that may reduce perspiration, perspiration odors, wetness resulting from perspiration, or combinations thereof. In some examples, the active antiperspirant ingredient may be a salt that reduces perspiration by diffusing through the sweat ducts of sweat glands. The salts may then combine with proteins to plug sweat ducts.

Examples of active antiperspirant ingredients include astringent water-soluble inorganic and organic salts of aluminum, zirconium and zinc or any mixtures of these salts. Examples of active antiperspirant ingredients include, aluminum zirconium techlorohydrex, aluminum halides, aluminum chlorohydrates, aluminum hydroxyhalides, zirconyl oxyhalides, zirconyl hydroxyhalides, aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, and combinations thereof.

While the above examples have been described with reference to specific types of active antiperspirant ingredients, any appropriate antiperspirant ingredient may be used in accordance with the principles described herein. For example, the active antiperspirant ingredients may be used to prevent perspiration and perspiration odors, inhibit the formation of perspiration and perspiration odors, or otherwise contribute to reducing perspiration. Further, the active antiperspirant ingredients may include multiple types of active antiperspirant ingredients that reduce perspiration and perspiration odors. In such examples, each of the active antiperspirant ingredients may perform different functions, perform overlapping functions, perform the same functions, or combinations thereof.

The antiperspirant composition (100) may include the active antiperspirant ingredient in an amount that provides an antiperspirant effect. For example, the active antiperspirant ingredient may form between 50.0 and 70.0 weight percent of the antiperspirant composition (100), such as 62.5 weight percent.

The water-phase component of the antiperspirant composition (100) may also include a carrier ingredient to disperse the active antiperspirant ingredient throughout the water-phase component. The carrier ingredient may include any ingredient that disperses the active antiperspirant ingredient throughout the water-phase component. For example, the carrier ingredient may be propylene glycol which disperses the active antiperspirant salt throughout the water-phase component. Another example of a carrier ingredient is dipropylene glycol. The carrier ingredient may form between 3.0 and 12.0 weight percent of the antiperspirant composition (100). More specifically, the carrier ingredient may form between 6.0 and 7.0 weight percent of the antiperspirant composition (100), such as 6.5 weight percent.

In some examples, the water-phase component may include a polymeric solubilizer. The polymeric solubilizer may aid in the emulsification of the water-phase component and the oil-phase component. For example, the polymeric solubilizer may have an amphiphilic chemical structure. An amphiphilic chemical structure may refer to a chemical structure with a hydrophilic end and a lipophilic end. The hydrophilic end may attract water-phase components and the lipophilic end may attract oil-phase components. Accordingly, the polymeric solubilizer may attract and stabilize both water-phase components and oil-phase components. Accordingly, the polymeric solubilizer may be any polymer that has an amphiphilic chemical structure. Examples of polymeric solubilizers include polyvinyl caprolactam, polyvinyl acetate, a polyethylene glycol graft copolymer, or combinations thereof. Adding the polymeric solubilizer as described herein may be beneficial in that it may enhance the wash-off capability of the antiperspirant active ingredient from the fabric. An increased wash-off capability may be beneficial in that it reduces the environment in which the active antiperspirant ingredient interacts with other compounds (such as sweat, sebum soils, and laundry detergent) to form precipitates on the fabric. In some examples, the polymeric solubilizer may form 0.01 to 1.0 weight percent of the water-in-oil emulsion, 0.05 to 0.2, for example. In some examples, the water-phase component may form between 65.0 and 72.0 weight percent of the antiperspirant composition (100), such as 69.0 to 69.10 weight percent.

The antiperspirant composition (100) may also include an oil-phase component. The oil-phase component may include a mineral clay emulsifier to stabilize the water-phase component and the oil-phase component. The mineral clay emulsifier may refer to any mineral clay mixture that includes a number of mineral clays. For example, the mineral clay mixture may include sepiolite rods and montmorillonite platelets. The sepiolite rods may form 75.0 to 85.0 weight percent of the mineral clay mixture, such as 80.0 weight percent and the montmorillonite platelets may form 15.0 to 25.0 weight percent of the mineral clay mixture. Mineral clay emulsifiers may include mineral clays such as quaternium-90 sepiolite, quaternium-90 montmorillonite, or combinations thereof. In some examples, the mineral clay mixture may be treated by a quaternary ammonium compound.

In one example, the mineral clay emulsifier may encapsulate the active antiperspirant ingredient. For example, mineral clay particles may encapsulate the active antiperspirant ingredient, and prevent the active antiperspirant ingredient from reacting with a detergent, sweat, sebum soils, or other ingredients in the antiperspirant composition (100). More specifically, the rod and platelet structure of a mineral clay emulsifier of sepiolite and montmorillonite may prevent an interaction between detergent and fabric. Accordingly, the encapsulant mineral clay emulsifier may prevent the formation of precipitates on fabric by reducing the probability of an interaction of the active antiperspirant ingredient with detergent, sweat glands, sebum soils, other ingredients within the antiperspirant composition (100) as the active antiperspirant ingredient is not in contact with these compounds. The use of a mineral clay as an emulsifier may be a unique aspect of the antiperspirant composition (100) as general practice in the industry relies on other types of emulsifiers such as dimethicone copolyol. A mineral clay emulsifier may be beneficial in that the solid clay particles may easily wash off, thus the amount of time the active antiperspirant ingredient is in contact with the fabric is reduced.

In some examples, the mineral clay emulsifier may form between approximately 0.25 to 3.0 weight percent of the water-in-oil emulsion, 0.6 to 0.9 weight percent for example. The amount of the mineral clay emulsifier in the antiperspirant composition (100) may be selected based upon the particular viscosity desired for the antiperspirant composition (100). Specific examples of commercially available mineral clay mixtures that may be used as emulsifiers include, but are not limited to, those sold under the name GARAMITE® by Southern Clay Products, Inc. of Gonzales, Tex.

The oil-phase component may also include a wetting agent to disperse the mineral clay emulsifier. For example, the wetting agent may disperse the mineral clay emulsifier throughout the oil-phase component. In some examples, the wetting agent may refer to any agent that disperses the mineral clay emulsifier throughout the oil-phase component of the antiperspirant composition (100). The wetting agent may comprise propylene carbonate. In some examples, the amount of wetting agent present in the oil-phase component may be relative to the amount of mineral clay emulsifier. For example, a ratio of wetting agent to the mineral clay emulsifier may be between 5.5 to 9.0 weight percent, such as 6.8 weight percent. The inclusion of the wetting agent in the antiperspirant composition (100) may be unique in that general practice in the industry relies on other emulsifiers that don't implement a wetting agent. Including the wetting agent in the antiperspirant composition (100) may be beneficial in that it may more evenly distribute the mineral clay emulsifier throughout the oil-phase component, thus increasing the emulsification effect of the mineral clay emulsifier.

In addition to the elements described above, the oil-phase component may include other ingredients. For example, the oil-phase component may include an emollient, a carrier, and a number of structurants, among other ingredients to improve the antiperspirant effect, application aesthetics, or combinations thereof of the antiperspirant composition (100). In some examples, the oil-phase component may form between 25.0 and 35.0 weight percent of the antiperspirant composition (100), such as 29.5 weight percent.

The antiperspirant composition (100) may include other ingredients such as anti-bacterial additives, dyes, antioxidants, and moisturizers among other additive to achieve a desired purpose or function. Specifically, the antiperspirant composition (100) may include fragrances to provide the antiperspirant composition (100) with a pleasant smell. In some examples, the fragrance may form between 0.1 and 3.0 weight percent of the antiperspirant composition (100), such as 1.5 weight percent.

The antiperspirant composition (100) may include a deodorant to reduce odors emanating from perspiration. Examples of deodorant additives include any agent to neutralize, suppress, or mask perspiration odor.

In addition to the aforementioned components, further additives may be included in the antiperspirant composition for various purposes including additives that cause the antiperspirant composition to exhibit long-lasting fragrance, odor protection, bacteria control, and/or another desired purpose and/or function. Specific examples of additional such additives include, but are not limited to, fragrances, including encapsulated fragrances; skin conditioners; dyes; pigments; preservatives; antioxidants; moisturizers; and the like.

Tables (1)-(3) present an example of an antiperspirant composition (100) as described herein. More specifically, the antiperspirant composition (100) may include a water-phase component that includes the following ingredients in the corresponding weight percentages as indicated in Table (1).

TABLE (1)

| Ingredient | Weight Percent (%) |
|---|---|
| Aluminum Zirconium Tetrachlorohydrex | 62.5 |
| Propylene Glycol | 6.5 |

As used in Table (1), aluminum zirconium tetrachlorohydrex may be the active antiperspirant ingredient and propylene glycol may be the carrier of the active antiperspirant ingredient. The antiperspirant composition (100) may include an oil-phase component that includes the following ingredients in the corresponding weight percentages as indicated in Table (2).

TABLE (2)

| Ingredient | Weight Percent (%) |
|---|---|
| C12-15 Alkyl Benzoate | 8.25 |
| Cyclohexasiloxane | 8.283 |
| Low Molecular Weight Polyethylene | 10.942 |
| High Molecular Weight Polyethylene | 1.223 |
| Quaternium-90 Sepiolite, | 0.7500 |
| Quaternium-90 Montmorillonite | |
| Propylene Carbonate | 0.052 |

As used in Table (2), C12-15 alkyl benzoate may be an emollient, cyclohexasiloxane may be a carrier, low molecular weight polyethylene component A and high molecular weight polyethylene component B may be structurants, quaternium-90 sepiolite, quaternium-90 montmorillonite may be a mineral clay emulsifier and propylene carbonate may be a wetting agent. The low molecular weight polyethylene may have a melting point between 66 degrees Celsius and 73 degrees Celsius. The high molecular weight polyethylene may have a melting point between 75 degrees Celsius and 86 degrees Celsius.

Table (3) indicates an overall antiperspirant composition (100) including the water-phase component as indicated in Table (1) and the oil-phase component as indicated in Table (2).

As indicated in Table (3), the antiperspirant composition (100) may include a water-phase component as indicated in Table (1), an oil-phase component as indicated in Table (2) and a fragrance in the corresponding weight percentages as indicated in Table (3).

TABLE (3)

| Ingredient | Weight Percent (%) |
|---|---|
| Water-phase Component | 69.0 |
| Oil-phase Component | 29.5 |
| Fragrance | 1.5 |

Tables (4)-(6) present another example of an antiperspirant composition (100) as described herein. More specifically, the antiperspirant composition (100) may include a water-phase component that includes the following ingredients in the corresponding weight percentages as indicated in Table (4).

TABLE (4)

| Ingredient | Weight Percent (%) |
|---|---|
| Aluminum Zirconium Tetrachlorohydrex | 62.5 |
| Propylene Glycol | 6.5 |
| Polyvinyl Caprolactam, Polyvinyl | 0.10 |
| Acetate, Polyethylene Glycol Graft | |
| Polymer | |

As used in Table (4), aluminum zirconium tetrachlorohydrex may be the active antiperspirant ingredient, propylene glycol may be the carrier ingredient, and the polyvinyl caprolactam, polyvinyl acetate, polyethylene glycol graft polymer, or combinations thereof may form part of the polymeric solubilizer. The antiperspirant composition (100) may include an oil-phase component that includes the following ingredients in the corresponding weight percentages as indicated in Table (5).

TABLE (5)

| Ingredient | Weight Percent (%) |
|---|---|
| C12-15 Alkyl Benzoate | 8.15 |
| Cyclohexasiloxane | 8.283 |
| Low Molecular Weight Polyethylene | 10.942 |
| High Molecular Weight Polyethylene | 1.223 |
| Quaternium-90 Sepiolite, | 0.7500 |
| Quaternium-90 Montmorillonite | |
| Propylene Carbonate | 0.052 |

As used in Table (5), C12-15 alkyl benzoate may be an emollient, cyclohexasiloxane may be a carrier, low molecular weight polyethylene component A and high molecular weight polyethylene component B may be structurants, quaternium-90 sepiolite, quaternium-90 Montmorillonite may be a mineral clay emulsifier and propylene carbonate may be a wetting agent. The low molecular weight polyethylene may have a melting point between 66 degrees Celsius and 73 degrees Celsius. The high molecular weight polyethylene may have a melting point between 75 degrees Celsius and 86 degrees Celsius.

As indicated in Table (6), the antiperspirant composition (100) may include a water-phase component as indicated in Table (4), an oil-phase component as indicated in Table (5) and a fragrance in the corresponding weight percentages as indicated in Table (6).

TABLE (6)

| Ingredient | Weight Percent (%) |
|---|---|
| Water-phase Component | 69.0 |
| Oil-phase Component | 29.5 |
| Fragrance | 1.5 |

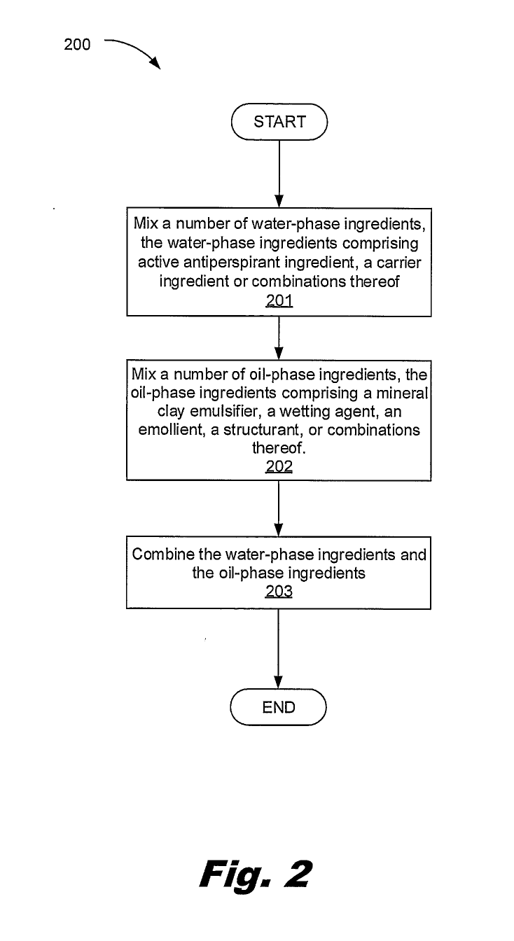

FIG. 2 is a flowchart of a method (200) for making a water-in-oil emulsion antiperspirant composition (100) according to the principles described herein. The antiperspirant composition can be made through other techniques, and the antiperspirant composition (100) is not limited to any particular method of making the same. The method (200) may include mixing (block 201) a number of water-phase ingredients. As described above, the water-phase ingredients may include an active antiperspirant ingredient, a carrier ingredient, or combinations thereof The water-phase ingredients may also include a polymeric solubilizer. As described above, including the polymeric solubilizer in the water-phase may be beneficial and unique in that it may enhance the ability of the active antiperspirant ingredient to wash off the fabric quicker, thereby reducing the ability for precipitates to form on the fabric.

The method (200) also includes mixing (block 202) a number of oil-phase ingredients. The oil-phase ingredients may include a mineral clay emulsifier, a wetting agent, an emollient, a number of structurants or combinations thereof. As described above, the oil-phase ingredient may include a mineral clay emulsifier that may encapsulate the active antiperspirant ingredient. A mineral clay emulsifier may be a unique aspect of the antiperspirant composition (100) as it is general practice in the field to use other types of emulsifiers. A mineral clay emulsifier that encapsulates the active antiperspirant ingredient may be beneficial in that it 1) prevents interaction between the active antiperspirant ingredient and laundry detergent, sweat, sebum, other ingredients in the antiperspirant composition (100) or combinations thereof and 2) washes off easier, thus giving the active antiperspirant ingredient less time for precipitate formation. The oil-phase ingredients and the water-phase ingredients may then be combined (block 203). Accordingly, a water-in-oil emulsion antiperspirant composition (100) is formed that may be beneficial in that it produces less white residue on a skin surface during application and it may also improve application aesthetics.

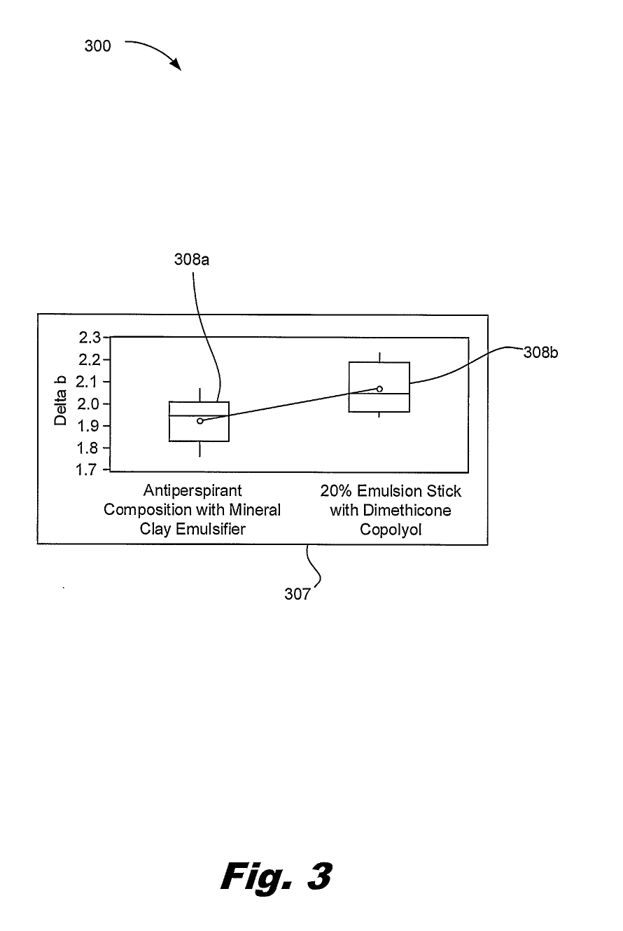

FIG. 3 is a diagram (300) illustrating stain formation using the antiperspirant composition (100) with a mineral clay emulsifier according to the principles described herein. More particularly, the diagram (300) may include a box plot (307) that indicates a visual perception of yellow staining on a fabric. The box plot indicates yellow staining after 4 wash cycles of the fabric. Yellow stain perception may be indicated on the y-axis as “delta b” which is a value measured by a colorimeter that indicates the presence of the color yellow. A less positive value of delta b indicates less yellow.

The x-axis of the box plot (307) may correspond to different antiperspirant compositions. As indicated in the box plot (307) an antiperspirant composition (100) with mineral clay emulsifier as described herein was tested against a 20% active emulsion stick that used dimethicone copolyol as an emulsifier. The box plot (307) may include a number of boxes (308a, 308b) that indicate the quartile amounts of yellow perceived when a fabric has been treated with a particular antiperspirant composition. For example, the first box (308a) may indicate the quartile ranges and the greatest and least amounts of yellow detected on a fabric treated with the antiperspirant composition (100) with a mineral clay emulsifier as described herein. By comparison, the second box (308b) may indicate the quartile ranges and the greatest and least amounts of yellow detected on a fabric treated with a 20% active emulsion antiperspirant stick that uses dimethicone copolyol as the emulsifier.

As indicated by the box plot (307) in FIG. 3, the antiperspirant composition (100) using a mineral clay emulsifier produces less yellow staining as compared to a comparable product that does not use a mineral clay emulsifier. More specifically, the greatest amount of yellow detected, the least amount of yellow detected, and each quartile measurement for the antiperspirant composition (100) with the mineral clay emulsifier is less than corresponding values for the antiperspirant stick using a dimethicone copolyol emulsifier. Thus, not only is the use of a mineral clay emulsifier unique, but it is measurably better than a dimethicone copolyol emulsifier.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant composition with a mineral clay emulsifier, comprising:
a water-in-oil emulsion comprising:
a water-phase component comprising an active antiperspirant ingredient and a carrier ingredient; and
an oil-phase component comprising a mineral clay emulsifier to stabilize the water-phase component and the oil-phase component.

2. The antiperspirant composition of claim 1, in which the water-in-oil emulsion is a pickering emulsion.

3. The antiperspirant composition of claim 1, in which the mineral clay emulsifier encapsulates the active antiperspirant ingredient.

4. The antiperspirant composition of claim 1, in which the mineral clay emulsifier comprises sepiolite, montmorillonite or combinations thereof.

5. The antiperspirant composition of claim 1, in which the mineral clay emulsifier forms between 0.25 to 3.0 weight percent of the water-in-oil emulsion.

6. The antiperspirant composition of claim 1, in which the water-phase component further comprises a polymeric solubilizer.

7. The antiperspirant composition of claim 6, in which the polymeric solubilizer comprises polyvinyl caprolactum, polyvinyl acetate, a polyethylene glycol graft copolymer or combinations thereof.

8. The antiperspirant composition of claim 6, in which the polymeric solubilizer forms 0.01 to 1.0 weight percent of the water-in-oil emulsion.

9. The antiperspirant composition of claim 1, further comprising a wetting agent to disperse the mineral clay emulsifier.

10. The antiperspirant composition of claim 9, in which the wetting agent comprises propylene carbonate.

11. The antiperspirant composition of claim 9, in which a ratio of the wetting agent to the mineral clay emulsifier is between 5.5 to 9.0 weight percent.

12. The antiperspirant composition of claim 11, in which the ratio is 6.8 weight percent.

13. An antiperspirant product with a mineral clay emulsifier comprising:
a container; and
an antiperspirant composition housed within the container and comprising:
an active antiperspirant ingredient disposed within a water-phase component of a water-in-oil emulsion;
a carrier ingredient disposed within the water-phase component;
a mineral clay emulsifier disposed within an oil-phase component of a water-in-oil emulsion; and
a wetting agent disposed within the oil-phase component, the wetting agent dispersing the mineral clay emulsifier within the oil-phase component.

14. The antiperspirant product of claim 13, further comprising a dye, a fragrance, an emollient, a structurant, or combinations thereof.

15. The antiperspirant product of claim 13, in which a ratio of the wetting agent to the mineral clay emulsifier is between 5.5 to 9.0 weight percent.

16. The antiperspirant product of claim 13, in which the mineral clay emulsifier comprises sepiolite, montmorillonite or combinations thereof.

17. The antiperspirant product of claim 13, in which the antiperspirant composition is configured to reduce precipitate formation on fabric.

18. The antiperspirant product of claim 13, in which the mineral clay emulsifier encapsulates the active antiperspirant ingredient.

19. A method for making a water-in-oil emulsion antiperspirant composition, the method comprising the steps of:
mixing an active antiperspirant ingredient with a carrier ingredient; and
mixing a number of oil-phase ingredients, the oil-phase ingredients comprising a mineral clay emulsifier, a wetting agent, an emollient, a structurant, or combinations thereof.

20. The method of claim 19, further comprising, mixing a polymeric solubilizer with the active antiperspirant ingredient and the carrier ingredient.
